(19) United States
(12) Patent Application Publication
(10) Pub. No.: US 2014/0163074 A1
(43) Pub. Date: Jun. 12, 2014

(54) Pharmaceutical composition for treating dermatological autoimmune diseases

(71) Applicant: AGON Pharma GmbH, Wendlingen (DE)

(72) Inventors: Günter Stephan, Stuttgart (DE); Charlie Farr, Vienna (AT)

(73) Assignee: AGON Pharma GmbH, Wendlingen (DE)

(21) Appl. No.: 14/095,616

(22) Filed: Dec. 3, 2013

Related U.S. Application Data
(62) Division of application No. 13/272,750, filed on Oct. 13, 2011, now abandoned.
Application No. 13/272,750 is a continuation of application No. PCT/EP2010/054979, filed on Apr. 15, 2010.

(30) Foreign Application Priority Data
Apr. 15, 2009 (DE) 10 2009 018 133.4

(51) Int. Cl. A61K 31/4439

(52) U.S. Cl. CPC A61K 31/4439
USPC 514/338

(57) ABSTRACT

There is proposed a pharmaceutical composition for treating dermatological autoimmune diseases, comprising a proton pump inhibitor.

The present invention relates to therapeutic compositions for treating dermatological autoimmune diseases, in particular for treating atopic eczema.

Atopic eczema is a skin disease from the immunological category of diseases. It is synonymously also called neurodermatitis, atopic dermatitis, endogenous eczema, chronic constitutional eczema, asthma eczema or Besnier's prurigo. About 1 to 3% of adults are affected by this disease in Germany, for example, with atopic eczema being assumed to occur 4 to 6 times more frequently since the middle of the 20^{th} century up until today. Children are most affected at about 10 to 20%; in about 60% of those affected, the disease occurs in the first year of life and disappears again in the course of childhood in about 30 to 50% of these patients.

The main symptoms of atopic eczema are red, scaly, sometimes also weeping eczema on the skin and a severe itchiness even lasting into the night. The face is primarily affected in babies and later the atopic eczema typically extends to the large joints, hands, feet and throat. The primary symptoms in adults are dry skin, lichenification and scratched lumps.

Currently, a predisposition is considered certain to be the primary cause of atopic eczema, i.e. a genetic disorder both of the epidermal and the immunological barrier function of the skin. This genetic predisposition leads to the disease symptoms in an interaction with environmental factors and psychological influences.

Like the causes, the applied forms of treatment are also very different, and not all treatment approaches are equally effective in all those affected. The focal point of symptomatic therapy is the basic care of the skin in the form of a topical treatment with ointments, creams or lotions, whereby a barrier function of the skin is stabilised and its sensitivity to irritations and the penetration of allergens is to be weakened. Urea-containing preparations, in particular, help to reduce the characteristic dryness of the skin. Further frequently used active ingredients are, for example, evening primrose oil, St. John's wort extract, zinc and dexpanthenol.

Immuno-suppressants or anti-inflammatory active ingredients are used for the topical treatment of more severe inflammatory symptoms. Glucocorticoids are most frequently used here, by means of which severe attacks are ameliorated or can be avoided with timely application. Depending on the type and severity of the symptoms, both highly effective glucocorticoids (Class 3) and also weak or medium-strength preparations (Classes 1 and 2) are available. In particular when glucocorticoids are applied over a large area, side effects may, however, occur in the form of thinning of the skin (atrophy), pigmentation disorders, thick hair growth (hypertrichosis), stretch marks and a partial suppression of the local immune system. A treatment with glucocorticoids should therefore only be short-term.

As an alternative to glucocorticoids, the immuno-suppressants tacrolimus and pimecrolimus, which can be applied locally, have been available for a few years. Both substances belong to the group of macrolides and act as calcineurin inhibitors. In contrast to glucocorticoids, they have no atrophying effects and are primarily used where the skin is very thin and an increased penetration risk therefore exists for the active ingredients (face, neck and genital area). Up until now, there has been no long-term experience as to whether these active ingredients contribute to the formation of tumours, so the US Pharmaceutical Authority FDA published a corresponding warning in 2005. The European Pharmaceutical Agency EMEA also limits the use to cases in which adequate therapy success cannot be achieved with glucocorticoids or the side effects do not allow glucocorticoid therapy.

Active ingredients for internal application are, in particular, antihistamines which can ameliorate the itching. This is mainly used in children so that they can fall asleep more easily and do not need to scratch so much. The internal application of cortisone and cyclosporin A is limited to the severe or most severe forms of atopic eczema because of the considerable side effects.

It has now been surprisingly found that proton pump inhibitors are effective in the treatment of atopic eczema and other dermatological autoimmune diseases.

The subject of the present invention is a pharmaceutical composition, comprising a proton pump inhibitor, for treating dermatological autoimmune diseases.

The invention therefore also relates to the use of a proton pump inhibitor for treating dermatological autoimmune diseases.

Proton pump inhibitors (PPIs) are active ingredients which were hitherto only used to treat diseases in which a reduction of the stomach acid secretion is indicated. This includes, in particular, the stomach ulcer (ulcus ventriculi), the duodenal ulcer (ulcus duodeni), reflux disease of the oesophagus (reflux oesophagitis) and Zollinger-Ellison-Syndrome. Proton pump inhibitors block the proton-potassium pump responsible for the secretion of stomach acid (H⁺/K⁺-ATpase) in the parietal cells of the stomach lining.

The proton pump inhibitor preferably comprises a substituted pyridylmethylsulfinyl benzimidazole or its pharmaceutically acceptable salt, in particular a compound having the following general Formula I:

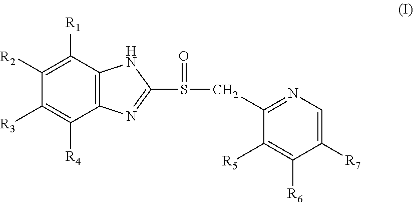

wherein R₁ to R₄ are the same or different and are in each case selected from hydrogen, alkyl, alkoxy, halogen, halogenalkoxy, alkylcarbonyl, alkoxycarbonyl, oxazolyl and trifluoroalkyl, or wherein adjacent groups R₁ to R₄ form ring structures, which can be further substituted; and wherein R₅, R₆ and R₇ are the same or different and are in each case selected from hydrogen, alkyl, optionally fluorine-substituted alkoxy, alkylthio, alkoxyalkoxy, dialkylamino, piperidino, morpholino, halogen, phenyl and phenylalkoxy.

The substituted pyridylmethylsulfinyl benzimidazoles, in the framework of their use as proton pump inhibitors, are so-called prodrugs, which are converted by the acid medium in the stomach into a sulfenamide, which, as the actual active substance, irreversibly inhibits the proton-potassium pump. They are therefore also called acid-sensitive proton pump inhibitors. In addition, these compounds also have an antibacterial effect against helicobacter pylori.

Typical representatives of the acid-sensitive proton pump inhibitors are, in particular omeprazole having the Formula II,

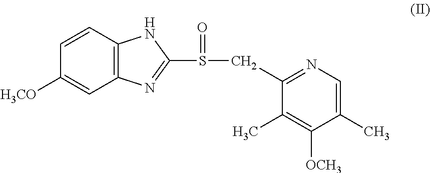 [SMILES: COc1ccc2[nH]c(S(=O)Cc3ncc(C)c(OC)c3C)nc2c1]

the pure (S)-enantiomer of which is also called esomeprazole,
lansoprazole having the Formula III,

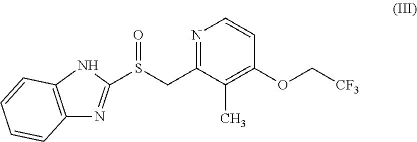 [SMILES: Cc1c(OCC(F)(F)F)ccnc1CS(=O)c1nc2ccccc2[nH]1]

and pantoprazole having the Formula (IV),

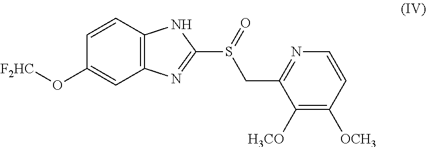 [SMILES: COc1ccnc(CS(=O)c2nc3cc(OC(F)F)ccc3[nH]2)c1OC]

The three active ingredients mentioned have an identical action mechanism in the inhibition of the proton pump, with the acid-induced conversion of pantoprazole taking place at significantly lower pH values than in the case of omeprazole and lansoprazole. The application to reduce the stomach acid secretion takes place systemically, in particular perorally by means of stomach acid-resistant tablets or capsules, or else intravenously.

The substituted pyridylmethylsulfinyl benzimidazoles belong to the most frequently prescribed medications in the gastrointestinal area, with side effects, when taken orally, being rare to very rare according to the present state of knowledge.

In the scope of the present invention, the proton pump inhibitor is therefore preferably selected from omeprazole, esomeprazole, lansoprazole, pantoprazole, the pharmaceutically acceptable salts thereof, and mixtures thereof. Pharmaceutically acceptable salts comprise, in particular, sodium and magnesium salts of the compounds mentioned.

The dermatological autoimmune diseases which can be effectively treated with proton pump inhibitors in the scope of the present invention comprise, in particular, the above-described atopic eczema, but are not limited thereto. A further indication is, for example, psoriasis, which is also an inflammatory reaction of the skin, which is inter alfa to be attributed to a genetic predisposition.

The pharmaceutical composition according to the present invention cannot only be applied in humans, but can also be used for the treatment of dermatological autoimmune diseases in animals. Summer eczema in horses should be mentioned here, in particular, for which similar causes are suspected as for atopic eczema in humans.

The pharmaceutical composition according to the invention is advantageously intended for topical application, i.e. for external application on the affected areas of the skin. In general, lesser side effects are to be assumed with a topical application than with a systemic administration, as a high concentration of active ingredient is only present in the corresponding target area. The risk of side effects in the known proton pump inhibitors which is already small in systemic administration, is therefore negligible with a topical application.

The pharmaceutical composition may basically be single-phase or multi-phase systems, i.e. homogeneous systems or emulsions which, depending on the type of basic substances or carrier substances used, are called ointments, creams, gels or lotions. According to a preferred embodiment of the invention, the pharmaceutical composition is present in the form of an ointment, i.e. as a single-phase system with a lipophilic carrier substance.

The pharmaceutical composition according to the present invention preferably comprises 0.1 to 20% by weight of the proton pump inhibitor, more preferably 0.3 to 10% by weight, and most preferably 1 to 5% by weight. Good results can generally already be achieved with active ingredient concentrations at the lower end of this range, in other words, for example, with an active ingredient concentration of about 1% by weight.

It is advantageous if the pharmaceutical composition comprises a lipophilic carrier substance as the basic ointment substance. The lipophilic carrier substance is preferably selected from synthetic and mineral waxes, fats and oils and synthetic derivatives thereof, as well as mixtures thereof. Examples of synthetic or mineral carrier substances are polyethylene glycols, aliphatic hydrocarbons (for example various paraffins and petroleum jelly) and silicones, which are in each case available in different molecular weight ranges.

Alternatively or additionally, the pharmaceutical composition may also comprise a lipophilic carrier substance, which is selected from animal and vegetable waxes, fats and oils and their hydrogenated or partially hydrogenated derivatives, as well as mixtures thereof, such as, for example beeswax, lanolin or plant oils.

The pharmaceutical composition is preferably substantially anhydrous, as the pyridylmethylsulfinyl benzimidazoles which can be used as proton pump inhibitors are not only acid-sensitive, but also generally water-sensitive. In particular, the above-described, acid-catalysed conversion into the corresponding sulfenamides is not desired in the scope of the application according to the present invention. By using anhydrous systems, the stability and durability of the pharmaceutical composition according to the invention can therefore be increased.

On the other hand, it has been shown that the pharmaceutical composition according to the invention can also be formulated as a two-phase system with a water fraction of up to 40% by weight with an adequate stability. The pharmaceutical composition, according to this further preferred embodiment of the invention, is a water-in-oil emulsion or an oil-in-water emulsion. In this case, the composition may also comprise one or more emulsifiers.

By adding suitable auxiliary substances, the acid-sensitivity of the proton pump inhibitors can additionally be taken into consideration. In order to adjust a pH of the carrier substance used in the alkaline range (preferably in the range of about 8.5 to 9.5), the pharmaceutical composition advantageously comprises an alkaline substance (for example an alcoholic potassium hydroxide solution), preferably in a quantity of 0.1 to 5% by weight.

It is also advantageous if the pharmaceutical composition comprises one or more buffering agents, preferably in a quantity of up to 5% by weight. This may be an anhydrous composition, in particular lipophilic amines, such as, for example stearylamine, which are soluble in a lipophilic carrier substance and are used to buffer protons, or in a two-phase system, for example, trometamol.

To bind possibly present residual water in an anhydrous pharmaceutical composition, the latter may comprise silicon dioxide, preferably in a quantity of 0.1 to 3% by weight.

In order to prevent oxidative destruction of the proton pump inhibitor and to increase the stability of the pharmaceutical composition, the latter may additionally comprise one or more lipophilic antioxidants, preferably in a quantity of up to 5% by weight, in particular from 0.01 to 2% by weight. Suitable lipophilic antioxidants are, for example, vitamin E, butylhydroxytoluene (BHT), butylhydroxyanisole (BHA) and ascorbyl palmitate.

In order to ensure an alkaline environment in the pharmaceutical composition, alternatively or in addition to the above-described measures, it is advantageous if a conditioning with an alkaline medium is carried out.

The subject of the present invention is therefore also a method for producing a pharmaceutical composition, comprising a proton pump inhibitor, for topical application, wherein the proton pump inhibitor is mixed with a lipophilic carrier substance and this is then treated with an alkaline medium. The pharmaceutical composition, in this case, is an ointment with the lipophilic carrier substance as the basic ointment substance, as was described above.

The alkaline medium, with which the lipophilic carrier substance is treated, preferably comprises an aqueous alkaline solution, in other words, for example, an aqueous sodium hydroxide solution or potassium hydroxide solution. Furthermore, the alkaline medium may comprise one or more stabilisers and/or antioxidants, such as, for example, sodium disulfide or sodium EDTA.

It is favourable if the lipophilic carrier substance is boiled out with the alkaline medium. The alkaline medium is then preferably removed.

The above-described auxiliary substance(s) can be added to the lipophilic carrier substance before or after the treatment with the alkaline medium.

The method for producing the pharmaceutical composition is favourably carried out completely or partially under a protective gas atmosphere, for example nitrogen or argon. An oxidisation of the proton pump inhibitor can thereby be avoided or at least reduced. In particular, the cooling of the lipophilic carrier substance after boiling out with the alkaline medium and the further processing preferably take place under a protective gas atmosphere.

Filling with the pharmaceutical composition is also preferably carried out under a protective gas atmosphere. Suitable containers comprise, in particular, tubes made of aluminium, plastics material or a composite material, for example aluminium tubes with a septum, polyfoil tubes (plastics material/aluminium/plastics material), multiplex composite tubes or plastics material containers produced by the blow-seal method.

The tubes or other containers for the pharmaceutical composition may have an inner coating, which contains antioxidants as additional product protection.

According to a further embodiment of the invention, as an alternative form of administration of the pharmaceutical composition, it may also be provided that the proton pump inhibitor or the composition is integrated into a polymer film, which is applied to the affected point on the skin. It is particularly advantageous here if the active ingredient is continuously dispensed from the polymer film.

These and further advantages of the invention will be described in more detail with the aid of the following examples.

### EXAMPLES

### Production of Pharmaceutical Compositions with a Proton Pump Inhibitor for Topical Application

### Formulations 1 to 4: Anhydrous System

Various pharmaceutical compositions were produced according to the present invention (preparations 1 to 4) in the form of ointments. All the compositions contained, as the proton pump inhibitor, micronised omeprazole, and various mixtures of lipophilic carrier substances as the basic ointment substance (petroleum jelly, various paraffins, Plastibase DAC, medium-chain triglycerides and beeswax). Furthermore, the compositions contained oil-soluble vitamin E and butylhydroxytoluene as lipophilic antioxidants and optionally stearylamine as the lipophilic buffering agent.

The precise composition of the formulations 1 to 4 is given in the following Table 1, in which all the data are to be understood as % by weight.

TABLE 1

| | Formula- | Formula- | Formula- | Formula- |
|---|---|---|---|---|
| | tion 1 | tion 2 | tion 3 | tion 4 |
| Omeprazole | 1.00 | 1.00 | 1.00 | 1.00 |
| Petroleum Jelly | 82.98 | 72.98 | 82.98 | — |
| Paraffin subliquidum | 5.00 | — | — | — |
| Paraffin Oil | — | — | 10.00 | 9.00 |
| Hard Paraffin | — | — | 5.00 | — |
| Plastibase DAC | — | — | — | 88.98 |
| Medium-chain | 5.00 | 15.00 | — | — |
| triglycerides | | | | |
| Beeswax | 5.00 | 8.00 | — | — |
| Vitamin E | 1.00 | 1.00 | 1.00 | 1.00 |
| Butylhydroxytoluene | 0.02 | 0.02 | 0.02 | 0.02 |
| Stearylamine | — | 2.00 | — | — |

Possible examples of further antioxidants are butylhydroxyanisole (0.01 to 0.5% by weight) and ascorbyl palmitate (0.01 to 0.2% by weight).

### Formulation 5: Water-in-Oil Emulsion

A further pharmaceutical composition according to the present invention (Formulation 5) was produced in the form of a two-phase system (water-in-oil emulsion). The precise composition is given in the following Table 2, in which all the data are to be understood as % by weight.

TABLE 2

| | Formulation 5 |
|---|---|
| Paraffin subliquidum | 5.0 |
| Lanolin | 8.0 |
| Petroleum Jelly | 40.0 |
| Medium-chain triglycerides | 7.5 |
| Vitamin E Acetate | 5.0 |
| Omeprazole | 1.0 |
| Butylhydroxytoluene | 0.2 |
| Water | 33.0 |
| Trometamol | 0.3 |

For production, the lipophilic carrier substances (paraffin subliquidum, lanolin, petroleum jelly and the medium-chain triglycerides) were firstly melted at 80° C. The trometamol was dissolved in the water and incorporated in the lipophilic carrier substances and homogenised. After cold stirring to about 25 to 30° C., the omeprazole dissolved or suspended in the vitamin E acetate, and butylhydroxytoluene were incorporated.

### Treatment of Dermatological Autoimmune Diseases

The effect of the proton pump inhibitor omeprazole in the form of a 1% by weight ointment according to the above Formulation 5 was investigated in ten patients from 10 to 35 years old, who had suffered from atopic eczema for several years and had acute symptoms.

The treatment took place by applying the ointment to the affected areas of the skin, in particular in the area of the elbows and hollows of the knees, once to three times daily, depending on the severity of the symptoms.

It was possible to achieve a significant improvement in the symptoms by means of the treatment. There was a reduction in itching, a stabilisation of the barrier function of the skin and a reduction in the reddened, weeping or bleeding eczemas. The healing of the atopic eczema generally occurred after a treatment time of 4 to 6 weeks.

Side effects were not observed.

This surprising and clear treatment success proves the effectiveness of proton pump inhibitors in the treatment of dermatological autoimmune diseases.

Furthermore, the efficacy of the composition according to the invention was also investigated in the treatment of summer eczema in horses. A healing of the sites affected, on which the ointment was applied, and a regrowth of the coat, was also demonstrated here in several animals.

1. Pharmaceutical composition, comprising a proton pump inhibitor, for treating dermatological autoimmune diseases.

2. Pharmaceutical composition according to claim 1, wherein the proton pump inhibitor comprises a substituted pyridylmethylsulfinyl benzimidazole or a pharmaceutically acceptable salt thereof.

3. Pharmaceutical composition according to claim 2, wherein the proton pump inhibitor is selected from omeprazole, esomeprazole, lansoprazole, pantoprazole, pharmaceutically acceptable salts thereof, and mixtures thereof.

4. Pharmaceutical composition according to any one of the preceding claims, wherein the dermatological autoimmune disease comprises atopic eczema.

5. Pharmaceutical composition according to any one of the preceding claims, wherein the dermatological autoimmune disease comprises psoriasis.

6. Pharmaceutical composition according to any one of the preceding claims for topical application.

7. Pharmaceutical composition according to any one of the preceding claims in the form of an ointment.

8. Pharmaceutical composition according to any one of the preceding claims, comprising 0.1 to 20% by weight of the proton pump inhibitor.

9. Pharmaceutical composition according to claim 8, comprising 0.3 to 10% by weight of the proton pump inhibitor.

10. Pharmaceutical composition according to claim 9, comprising 1 to 5% by weight of the proton pump inhibitor.

11. Pharmaceutical composition according to any one of the preceding claims, further comprising a lipophilic carrier substance.

12. Pharmaceutical composition according to any one of the preceding claims, wherein the lipophilic carrier substance is selected from synthetic and mineral waxes, fats and oils and their synthetic derivatives, and mixtures thereof.

13. Pharmaceutical composition according to any one of the preceding claims, wherein the lipophilic carrier substance is selected from animal and vegetable waxes, fats and oils and their hydrogenated or partially hydrogenated derivatives, and mixtures thereof.

14. Pharmaceutical composition according to any one of the preceding claims, wherein the pharmaceutical composition is substantially anhydrous.

15. Pharmaceutical composition according to any one of claims 1 to 13, wherein the pharmaceutical composition is a water-in-oil emulsion or an oil-in-water emulsion.

16. Pharmaceutical composition according to any one of the preceding claims, further comprising 0.1 to 5% by weight of an alkaline substance.

17. Pharmaceutical composition according to any one of the preceding claims, further comprising up to 5% by weight of one or more buffering agents.

18. Pharmaceutical composition according to any one of the preceding claims, further comprising up to 5% by weight of one or more lipophilic antioxidants.

19. Method for producing a pharmaceutical composition, comprising a proton pump inhibitor, for topical application, wherein the proton pump inhibitor is mixed with a lipophilic carrier substance and this is then treated with an alkaline medium.

20. Method according to claim 19, wherein the alkaline medium comprises an aqueous alkaline solution

21. Method according to claim 19 or 20, wherein the alkaline medium comprises one or more stabilisers and/or antioxidants.

22. Method according to any one of claims 19 to 21, wherein the lipophilic carrier substance is boiled out with the alkaline medium.

23. Method according to any one of claims 19 to 22, wherein the alkaline medium is removed after the treatment of the lipophilic carrier substance.

24. Method according to any one of claims 19 to 23, wherein one or more auxiliary substances are added to the lipophilic carrier substance before or after the treatment with the alkaline medium.

25. Method according to any one of claims 19 to 24, wherein the method is completely or partly carried out under a protective gas atmosphere.

26. Method according to any one of claims 19 to 25, wherein the pharmaceutical composition is filled under a protective gas atmosphere.

27. Method according to any one of claims 19 to 26, wherein the pharmaceutical composition is filled into tubes made of aluminium, plastics material or composite material.
